(19) United States
(12) Patent Application Publication
(10) Pub. No.: US 2020/0289233 A1
(43) Pub. Date: Sep. 17, 2020

(54) Light Carrier Mounting Clip for a Surgical Cannula

(71) Applicant: Kim A. Marsh, Naples, FL (US)

(72) Inventor: Kim A. Marsh, Naples, FL (US)

(21) Appl. No.: 16/711,784

(22) Filed: Dec. 12, 2019

Related U.S. Application Data
(60) Provisional application No. 62/778,564, filed on Dec. 12, 2018.

(51) Int. Cl. A61B 90/35; A61B 17/34; A61B 17/00

(52) U.S. Cl. CPC A61B 90/35; A61B 17/3421; A61B 2017/3484; A61B 2017/00477; A61B 2017/00238; A61B 17/00234

(57) ABSTRACT

A mounting clip is provided for releasably securing a light carrier to a surgical cannula. The clip includes a resilient, axially split sleeve having an opposing pair of split edges that extend between opposing circumferential ends of the sleeve. The sleeve carries a retaining bracket for securing a light carrier to the sleeve. The sleeve is resiliently compressed and inserted into a surgical cannula such that a light carrier held by the retaining bracket of the sleeve illuminates the interior of the cannula and a surgical site in which the cannula is being used.

### RELATED APPLICATION

This application claims the benefit of U.S. Provisional Application No. 62/778,564 filed Dec. 12, 2019.

### FIELD OF THE INVENTION

This invention relates to a mounting clip that securely and effectively attaches a light carrier to a surgical cannula or a tube of the type used for spinal surgery and other surgical and medical procedures.

### BACKGROUND OF THE INVENTION

Surgeons and other medical personnel commonly employ a tubular surgical instrument known as a cannula during spinal surgery and other types of surgical/medical procedures. The surgical cannula is especially effective for use during minimally invasive spinal surgery and permits the surgeon to introduce surgical instruments into the patient's body so that surgery may be effectively performed on the spine or other part of the body being treated.

Providing adequate lighting for minimally invasive spinal and other types of orthopedic surgery can be problematic. It can be particularly difficult to provide adequate illumination when working through a surgical cannula. Improving illumination would facilitate such surgery and contribute to improved surgical outcomes.

### SUMMARY OF THE INVENTION

It is therefore an object of the present invention to provide a mounting clip that allows a light carrier of the type suited for illuminating surgical and medical procedures to be effectively and securely attached to a surgical cannula so that significantly improved illumination is provided for spinal and orthopedic surgeries and other medical procedures that utilize such cannulas.

It is a further object of this invention to provide a light carrier mounting clip that is adapted to be quickly, reliably and securely attached to surgical cannulas having an assortment of diameters and lengths for addressing different surgical applications.

It is a further object of this invention to provide a light carrier mounting clip for a surgical cannula that features a relatively simple construction and which is easy to install and utilize for various surgical and medical purposes.

It is a further object of this invention to provide a mounting clip that permits a surgical or medical light carrier to be quickly, securely and effectively mounted to surgical cannula and other types of tubular surgical and medical instruments as required.

It is a further object of this invention to provide a mounting clip that that is especially effective for mounting a blade-type light carrier to a surgical cannula.

It is a further object of this invention to provide for a mounting clip for a light carrier that is effective for improving lighting in all types of medical procedures involving the use of a surgical cannula or other tubular surgical instrument.

This invention features a light carrier mounting clip for a surgical cannula. The clip includes a resilient split sleeve or ring that is axially or longitudinally split between opposing circumferential ends of the sleeve to provide the sleeve with split edges that extend fully between the opposing circumferential ends of the sleeve. The longitudinal split in the sleeve allows the sleeve to be resiliently expanded and contracted diametrically. Contracting the sleeve enables the sleeve to be inserted into and retained within the central channel of a surgical cannula. Diametrically expanding the sleeve allows the sleeve to be wrapped about the exterior surface of a cannula. A retaining bracket for holding a light carrier is mounted to an inside circumferential surface of the sleeve. The retaining bracket has a slot that extends transversely through the sleeve. The slot receives the light carrier and holds the light carrier releasably in place within the sleeve to illuminate surgical procedures being performed through the cannula.

In a preferred embodiment, the split edges of the sleeve may be separated by a gap that is closed to diametrically contract the sleeve. Alternatively, the split edges of the sleeve may overlap one another. In such cases the edges are spread apart to diametrically expand the sleeve.

One or more mounting tabs may be secured to the sleeve proximate one of the circumferential ends of the sleeve. Such tabs extend radially outwardly from the sleeve to support the sleeve in place within the surgical cannula.

### BRIEF DESCRIPTION OF THE DRAWINGS

Other objects, features and advantages will occur from the following description of a preferred embodiment and the accompanying drawings, in which:

FIG. 1 is an elevational side view of a preferred light carrier mounting clip in accordance with this invention;

FIG. 2 is an elevational enlarged end view of the mounting clip of FIG. 1;

FIG. 3 is a top view of a representative tab employed by the mounting clip to support the clip within the cannula;

FIG. 4 is a side elevational view of the support tab;

FIG. 5 is an exploded top view of a surgical cannula, a mounting clip in accordance with this invention, and a surgical light carrier depicted adjacent to one another and prior to operable interconnection of those components for use in accordance with this invention;

FIG. 6 is an alternative exploded side view of the surgical cannula, the light carrier and the light carrier mounting clip;

FIG. 7 is a perspective view of the surgical cannula in which the mounting clip of this invention is utilized;

FIG. 8 is a top plan view of the mounting clip of this invention holding a surgical light carrier and interengaged with a surgical cannula in accordance with this invention;

FIG. 9 is an enlarged top plan view of the assembled components depicted in FIG. 8; and

FIG. 10 is an elevational side view showing the illuminated surgical cannula interengaged with a patient's spine during spinal surgery; the surgical cannula is mounted to conventional surgical support structure and the mounting clip operatively supports and positions a surgical light carrier within the surgical cannula to illuminate the surgery.

### DETAILED DESCRIPTION OF PREFERRED EMBODIMENTS

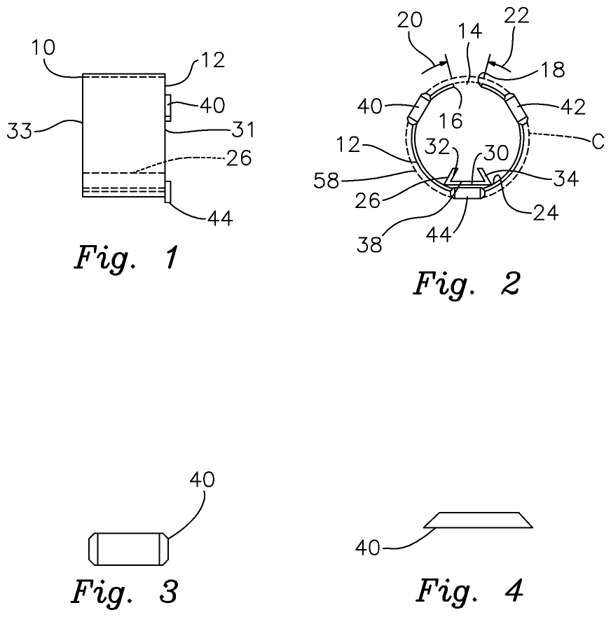

There is shown in FIGS. 1 and 2 a mounting clip 10 for securely holding and positioning a light carrier within a conventional surgical cannula so that a surgery or other medical procedure may be more effectively illuminated by the light carrier. It should be understood that tubular surgical cannulas and other tubular instruments of varying lengths and diameters are commonly utilized during spinal surgery, as well as other minimally invasive operations and medical procedures. The particular application for which the mounting clip is employed is not a limitation of this invention. Moreover, the mounting clip may be utilized with assorted types of surgical cannulas for analogous medical devices. Fundamentally, the mounting clip is designed to be releasably secured to the cannula and adapted to securely and releasably hold a surgical light carrier. Such light carriers may include an elongate blade composed of a light transmitting and projecting material. Various other types of light carriers may be supported within or otherwise on a surgical cannula by the mounting clip within the scope of this invention.

Mounting clip 10 includes a split annular band, ring or sleeve 12 preferably composed of a sturdy and durable, yet lightweight and resilient material. Typically, sleeve 12 is composed of a resilient or spring loaded metal or metal alloy. In alternative embodiments, a resilient plastic may be utilized. Sleeve 12 has a generally annular, cylindrical or tubular shape. As used herein, the term “sleeve” should be construed broadly to include all types of such shapes and analogous configurations. As best shown in FIG. 2, a longitudinal or axial split 14 is formed in sleeve 12, which split extends between the opposing ends 31, 33 of sleeve 12. This provides sleeve 12 with a split pair of opposing edges 16 and 18 that extend longitudinally or axially between opposing open circumferential ends 31 and 33 of the sleeve. Split 14 is effectively defined by the gap formed between edges 16 and 18. As used herein, “axial” and “axially” should be understood to mean that split 14 and edges 16 and 18 extend generally parallel to the central longitudinal axis of the sleeve. Sleeve 12 may be resiliently compressed by pushing the split transverse edges 16 and 18 together as indicated by arrows 20 and 22. This narrows the width of gap or split 14, which in turn, reduces the diameter of sleeve 12. Alternatively, split 14 may be widened by urging edges 16 and 18 of sleeve 12 apart from one another. This resiliently expands the diameter of sleeve 12.

An inside circumferential surface 24 of sleeve 12 supports a light carrier retaining bracket 26 or alternative component for retaining or holding a fiberoptic light carrier onto sleeve 12. The retaining bracket includes a generally flat base 30 and a pair of inwardly inclined side walls 32 and 34 that are attached unitarily and typically at least slightly resiliently to base 30. As a result, retaining bracket 26 defines a longitudinally or axially oriented slot 38 that extends longitudinally along the inside circumferential surface of sleeve 12 and generally parallel to the central axis of the sleeve between the opposite open ends of sleeve 12. Retaining bracket 26 is fastened permanently to the inner surface 24 of sleeve 12 by welding, pins, rivets or other fixed means of attachment. Various alternative forms of retaining brackets may be utilized to support a light carrier on the resilient sleeve in accordance with this invention.

Three support tabs 40, 42 and 44 are welded or otherwise permanently fastened to an open upper end 31 of sleeve 12. These tabs are spaced evenly about the circumference of sleeve 12. As shown by representative tab 40 in FIGS. 3 and 4, each tab includes a generally rectangular configuration having four rounded corners. Each tab also features two sides, which taper inwardly from back to front. Tabs 40, 42 and 44 facilitate positioning sleeve 12 at one end of a surgical cannula as described below.

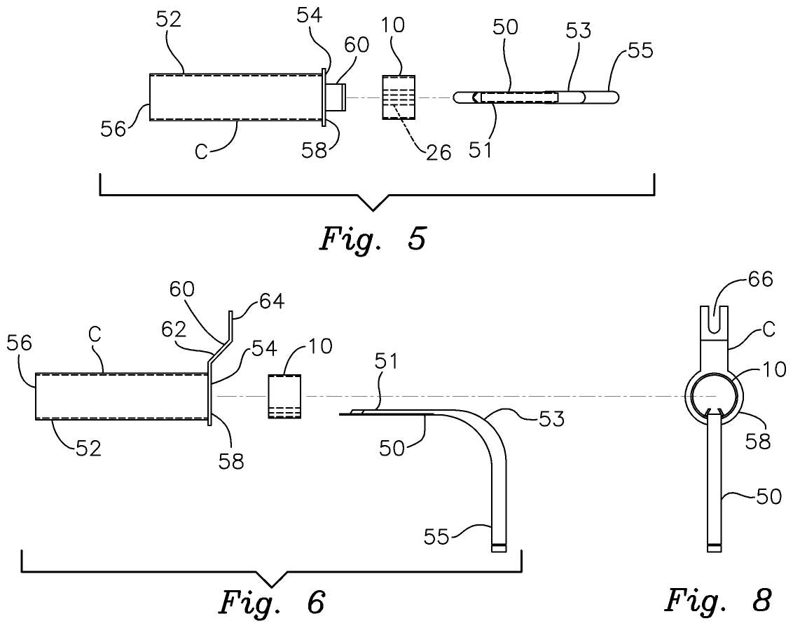

FIGS. 5 and 6 illustrate mounting clip 10 positioned between a surgical light carrier 50 and a surgical cannula C. The mounting clip is used to support and position the light carrier on the surgical cannula to improve illumination during surgery or other medical procedure.

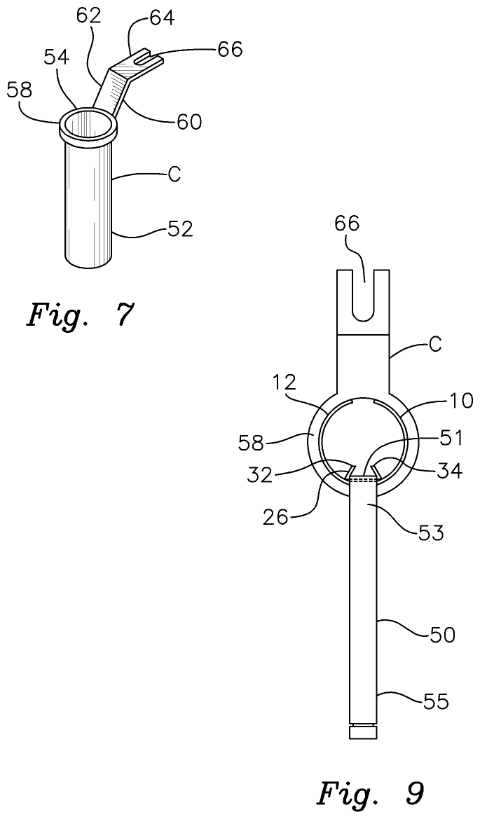

Surgical cannula C, which is shown alone in FIG. 7, includes an elongate tubular body 52 that has open upper and lower ends 54 and 56 respectively. An annular lip 58 surrounds open upper end 54 and a support arm 60 is attached integrally to lip 58. The support arm includes an upwardly inclined segment 62 and an upper plate 64. A notch 66 formed centrally in plate 64 is interengaged with a standard cannula holder, as shown more fully below in FIG. 10, such that the cannula may be securely mounted to a surgical table or other supportive structure in the operating room, in a manner that will be known to persons skilled in the art.

Light carrier 50 comprises a structure as shown, for example, in U.S. Pat. No. 10,064,613 (hereinafter U.S. Pat. No. '613) and Publication No. US2016/0361133 (hereinafter Pub. No. '133). The light carrier includes a generally flat blade 51 that is unitarily interconnected through a curved neck 53 to an inlet section 55. Each of blade 51, neck 53 and inlet section 55 includes a light conducting material. At least blade 50 also includes a light projecting material. Preferably, inlet section 55, neck 53 and blade 51 are composed of a single piece of light transmitting and projecting material of the type described in the foregoing references. The inlet section 55 is communicably and operably interconnected to an illumination source, again in the manner described in U.S. Pat. No. '613 and Pub. No. '133.

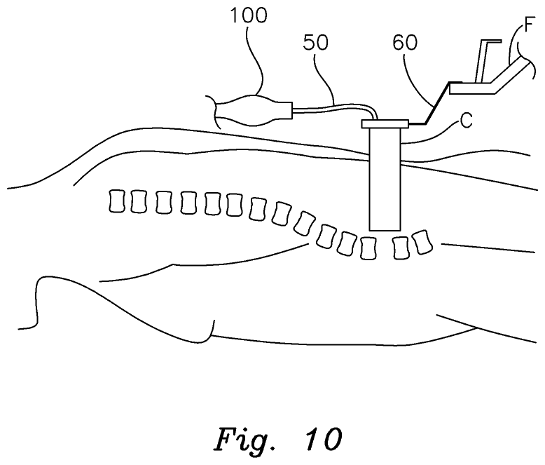

Light carrier 50, mounting clip 10 and surgical cannula C are operably interengaged in the manner shown in FIGS. 8 and 9. Light clip 10 may be initially inserted into the central channel formed through surgical cannula C. In particular, sleeve 12 of clip 10 is diametrically compressed by urging the opposing, split edges of the sleeve together, as shown by arrows 20, 22 in FIG. 2, and inserting the compressed sleeve into the central channel of the surgical cannula. It should be understood that in such versions, the spring biased sleeve is constructed such that it has a normal relaxed or unbiased diameter that is slightly greater than the diameter of the cannula's central channel. Sleeve 12 is compressed and clip 10 is inserted into cannula C until the previously described tabs (which are not shown in FIGS. 8 and 9) interengage circumferential lip 58 of cannula C. This limits insertion of clip 10 into cannula C. The normal spring bias of sleeve 12 urges the sleeve diametrically outwardly against the interior circumferential channel wall of cannula C. As a result, clip 10 snugly interengages and is held within cannula C.

Light carrier 50 is next interengaged with and supported by retaining bracket 26 of clip 10. More particularly, light projecting blade 51 is inserted into the previously described transverse slot 38 (FIG. 2) formed through bracket 26. See FIGS. 8 and 9. Blade 51 is held in place by opposing inclined arms 32 and 34 of bracket 26. The arms are spring biased or otherwise configured to securely engage and constrain movement of blade 51 within cannula C. This effectively positions blade 51 of light carrier 50 such that it extends at least partially, if not fully through the interior central channel of cannula C. The interconnected inlet section 55 and neck 53 of light carrier 50 are operatively interconnected to a standard fiberoptic light source by means not shown herein but described, for example, in U.S. Pat. No. '613. Light is thereby transmitted through light carrier 50 and projected from blade 51 into the cannula. As a result, improved illumination is provided through cannula C to the surgical site beyond the open lower end of the cannula. In alternative embodiments, the light carrier may be attached to clip 10 before the diametrically compressed sleeve is inserted into surgical cannula C.

FIG. 10 depicts a surgical operation and namely a minimally invasive spinal procedure, performed using a surgical cannula C equipped with the mounting clip of this invention and wherein a light carrier 50 is attached to the clip and thereby to the cannula. In particular, light carrier 50 is part of a handheld illumination system 100 of the type described in U.S. Pat. No. '613. In FIG. 10, surgical cannula C is introduced surgically into the patient's back such that the lower end of the cannula engages the patient's spine. As will be understood by persons skilled in the art, surgery upon the spine is typically performed by inserting surgical instruments through the central channel of cannula C. The cannula itself is supported by a conventional holder or frame F that is attached to the support arm 60 of cannula C. Light carrier 50 of surgical illumination assembly 100 is inserted into the cannula and held in place by mounting clip 10 as shown in FIGS. 8, 9 and 10. The generally flat light carrier blade 51 (FIGS. 5 and 6) is securely interengaged and held by bracket 26 (FIGS. 2, 8, and 9). The lower end of the light carrier blade extends into the central opening of cannula C (FIGS. 8-10) such that the patient's spine and the areas to be surgically operated upon are clearly illuminated. The mounting clip holds light carrier 50 securely in place to achieve consistent and much improved illumination of the area being surgically addressed.

It should be understood that in alternative embodiments, the light clip may be adapted to interengage an outer circumferential wall of a surgical cannula. In such cases, the sleeve of the clip should be manufactured and spring loaded to be normally smaller than the outer diameter of the applicable surgical cannula. The clip may then be diametrically expanded by opening the sleeve along the axial split so that the sleeve can be wrapped about the exterior surface of the cannula. The spring bias then urges the clip to diametrically contract against the outer cannula wall, which holds the clip in place. An alternatively configured light carrier may then be interengaged with a retaining bracket or other light carrier retaining structure on the clip and configured to extend into the central opening of the cannula so that the area on which surgery is being performed can be better illuminated.

Although the axial split formed in the mounting clip sleeve illustrated herein forms opposing longitudinally split sleeve edges that are spaced apart by a gap, in alternative embodiments, the split sleeve may extend for more than 360° between the respective split edges and be spring loaded such that the split edges of the sleeve overlap one another. Otherwise, in such versions the diameter of the sleeve is expanded and contracted as required in a manner analogous to that previously described.

Accordingly, this invention relates to a light carrier mounting clip that is particularly effective for supporting a light carrier on a surgical cannula. Although specific features of the invention are shown in some of the drawings and not others, this is for convenience only, as each feature may be combined with any and all of the other features in accordance with this invention.

What is claimed is:

1. A light carrier mounting clip for a surgical cannula, said clip comprising:
a sleeve composed of a resilient material and having a split formed axially therein between opposing ends of a said sleeve, which split enables said sleeve to be resiliently and diametrically adjusted for interengaging the surgical cannula; and
a retaining component attached to said sleeve for holding a light carrier to illuminate a surgical procedure being performed through the cannula.

2. The device of claim 1 in which said sleeve includes a pair of split edges that extend fully between said opposing ends of the sleeve.

3. The device of claim 1 in which said sleeve is resiliently and diametrically contracted for inserting into a central opening of the cannula and interengaging an interior wall of the cannula; said retaining component being attached to an interior cylindrical wall of said sleeve.

4. The device of claim 3 in which said retaining component includes an axially oriented slot for receiving the light carrier and holding the light carrier in place within the cannula.

5. The device of claim 1 in which said sleeve is resiliently and diametrically expanded for wrapping around and interengaging an exterior surface of the cannula.

6. The device of claim 1 in which said sleeve carries one or more positioning tabs proximate a said end of said sleeve for positioning said sleeve at one end of the cannula.

7. The device of claim 6 in which each said tab extends radially outwardly from said sleeve to support said sleeve in place within the surgical cannula.

8. The device of claim 2 in which said split edges are separated by a gap such that said sleeve may be diametrically contracted for inserting said sleeve into a central opening of the cannula.

9. The device of claim 2 in which said split edges overlap such that said sleeve is diametrically expanded for wrapping about and interengaging the outer surface of said cannula.

10. The device of claim 1 in which said retaining component includes a bracket having a slot for receiving the light carrier.

11. The device of claim 10 in which said retaining component includes a base that is secured to an inside circumferential surface of said sleeve and at least one resilient retaining arm attached to said base for securing the light carrier within said slot between said arm and said base.

12. A mounting clip for supporting a light carrier blade of a surgical lighting system on a surgical cannula, said clip comprising:
a sleeve composed of a resilient material and having a split formed axially therein between opposing ends of a said sleeve, which split enables said sleeve to be resiliently and diametrically adjusted for interengaging the surgical cannula; and
a retaining bracket attached to an inside circumferential surface of said sleeve and having a slot for receiving the light carrier blade and holding the light carrier blade to illuminate a surgical procedure being performed through the cannula.

13. A mounting clip for supporting a light carrier blade of a surgical lighting system on a surgical cannula, said clip comprising:
a sleeve composed of a resilient material and having a split formed axially therein between opposing ends of said sleeve, which split enables said sleeve to be resiliently and diametrically contracted for inserting into a central opening of the cannula and interior wall of the cannula; and
a retaining bracket attached to an inside circumferential surface of said sleeve and having a slot for receiving the light carrier blade and holding the light carrier blade within the central opening of the cannula to illuminate a surgical procedure being performed through the cannula.
